(19) United States
(12) Patent Application Publication
(10) Pub. No.: US 2012/0316499 A1
(43) Pub. Date: Dec. 13, 2012

(54) Fluid Delivery System

(76) Inventors: Erich George Brehm, Sunnyvale, TX (US); Erich Richard Brehm, Sunnyvale, TX (US); Marc Gerdisch, Indianapolis, IN (US)

(21) Appl. No.: 13/158,184

(22) Filed: Jun. 10, 2011

(51) Int. Cl. A61M 5/46

(52) U.S. Cl. 604/117

(57) ABSTRACT

A drug delivery system for delivering a drug may include a needle array to deliver the drug to the patient, a guide device to guide the needle array, and a base portion to connect the needle array to the guide device. The needle array may include a single row of needles. The guide device may include a concave end. The base portion may include a connector. The connector may include a luger type connector.

### FIELD OF THE INVENTION

This invention called “An Injection Sled” is generally in the field of devices for the administration of drugs and biological materials to patients. More particularly, this invention relates to needle arrays and methods for a precise depth and pattern control during insertion.

### BACKGROUND

Drug and biological materials require a delivery system that provides direct precise administration to the targeted tissue or organ. Without a means to control depth and pattern control, drugs and biological materials cannot be safely and accurately delivered to achieve the desired effect.

In summary, there is a need for a simple, effective, and economically desirable device for the administration of a variety of drug and biological material types to a patient while precisely controlling the depth, distance and pattern of the injection.

### SUMMARY

A drug and biological materials delivery system may include a needle array to deliver the drug to the patient, a guide or sled device to direct the needle array to a specific depth, distance and pattern, and a base portion to connect the needle array to the reservoir containing the drugs, biological materials, etc. to be delivered.

The needle array may include a single needle or row of needles.

The guide device may include a curved, roller or concave end.

The base portion may include a connector.

The connector may include a luger type connector.

The connector attaches to a tubing or directly to a reservoir containing drugs, biological materials, etc. to be delivered.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the drug delivery system of the present invention;

FIG. 2 illustrates a bottom view of the needle array, the guide device and the base portion of the drug delivery system of the present invention;

FIG. 3 illustrates a side view of the needle array, the guide device and the base portion of the drug delivery system of the present invention;

FIG. 4 illustrates a top view of the guide device and the base portion of the present delivery system of the present invention;

FIG. 5 illustrates a handle of the drug delivery system of the present invention which may be used with one or multiple needle array;

FIG. 6 illustrates another top view of the guide device and base portion of the drug delivery system of the present invention;

FIG. 7 illustrates a handle which is attachable to the guide device of the drug delivery system of the present invention;

FIG. 8 illustrates a top view of the guide device having a single needle;

FIG. 9 illustrates a top view of the guide device having multiple needles

FIG. 10 illustrates a side view of the guide device including a angled thumb handle;

FIG. 11 illustrates a side view of the guide device in a first position;

FIG. 12 illustrates a side view of the guide device in a second position.

### DETAILED DESCRIPTION

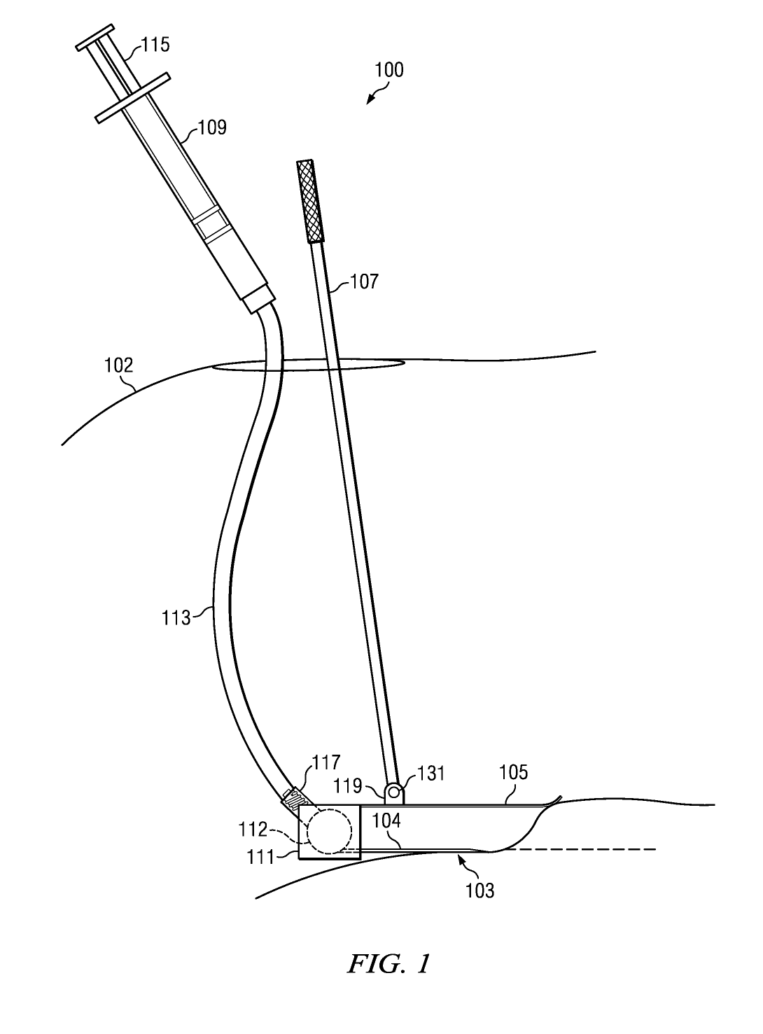

The present invention provides a drug delivery system 100 for the easy insertion of a needle or needles at a predetermined and controllable depth with respect to the top surface of the tissue of the patient. FIG. 1 illustrates a perspective view of the drug delivery system 100 of the present invention which may include a needle array 103, a guide device or sled 105 to guide the drug delivery system 100, a base portion 111 to interconnect the drug and biological materials source 109, the guide device 105 and the needle array 103. While the present invention discusses the delivery of drugs and biological materials by the delivery system 100, drugs can be interpreted to mean any fluid. The fluid source 109 may include a flexible tube 113 so that the drug source 109 may be in fluid communication with the base portion 111 and the needle array 103 and may include a plunger 115 so that a user may transmit the drug from the drug source 109 to a connector 117 of the base portion 111 and the needle array 103.

The guide device 105 may have a pivoting connection to the handle 107 so that the user can control the guide device 105 and the needle array 103. FIG. 1 illustrates the drug and biological materials delivery system 100 operating at a predetermined/controlled depth under the skin or tissue surface 102.

The needle array device 103 may include at least a single row of substantially parallel hollow cylinders 104 (needles), each having a substantially center passageway which extends from the proximate end of the hollow cylinder 104 to the distal end of the hollow cylinder 104 of the needle array device 103. The hollow cylinders may include a sharpened distal end. The needle array device 103 may include more than a single row of substantially parallel hollow cylinders 104 which may deliver a drug/material into the tissue at a predetermined/controlled spacing based upon the number of needles and the distance between the needles which may be a controlled design variable.

The base portion 111 may include a central cavity 112 which may be in fluid communication with each hollow cylinder 104 of the needle array 103 and maybe in fluid communication with the connector 117 in order to transmit the drug and/or biological materials from the base portion 111 to the needle array 103 and to the patient.

The guide device 105 which may be referred to as an injection sled may include a planar substrate which may be substantially rectangular, an oval or other appropriate shape and may include a first hinge portion 119 and a opposed second hinge portion 131 which may be connected to the surface of the guide device 105 in order to pivotably connect to the handle member 107.

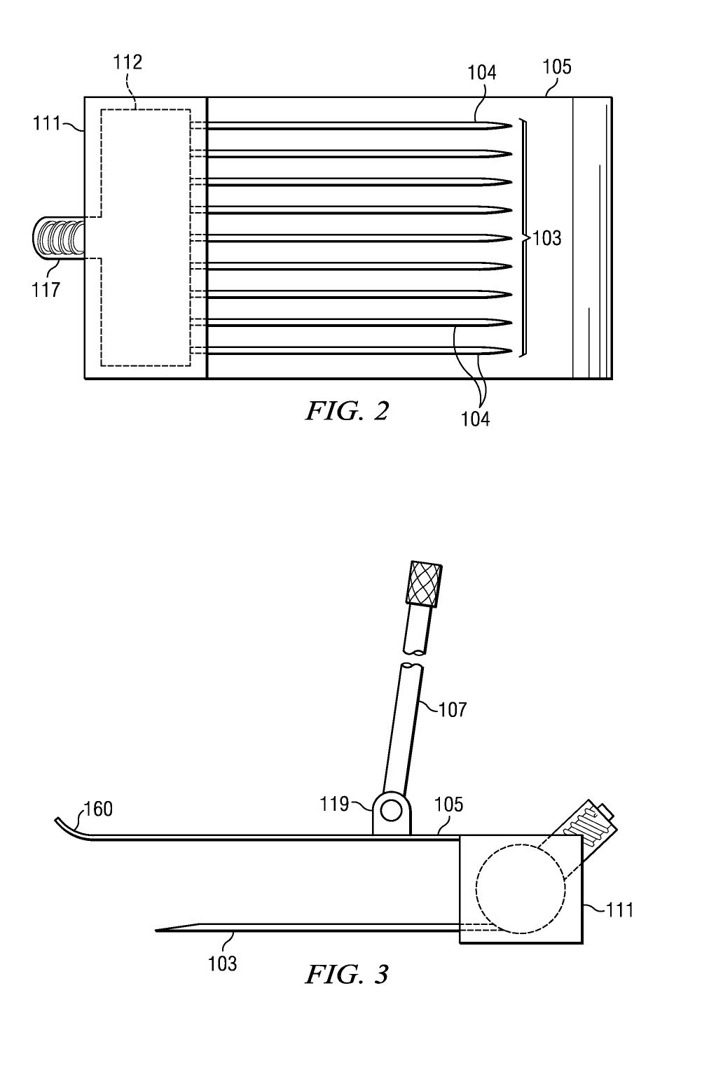

FIG. 2 illustrates a bottom view of the needle array 103, the base portion 111 to connect the guide device 105, the needle array 103 and the drug source 109, and a guide device 105 and illustrates the connector 117 to connect the flexible tube 119 to the base portion 111, the cavity 112 and the hollow cylinder 104.

FIG. 3 illustrates a side view of the needle array 103, the base portion 111 and the guide device 105 and illustrates the first hinge portion 119 and the handle 107. The distal end of the guide device 105 may include a concave curved portion 106.

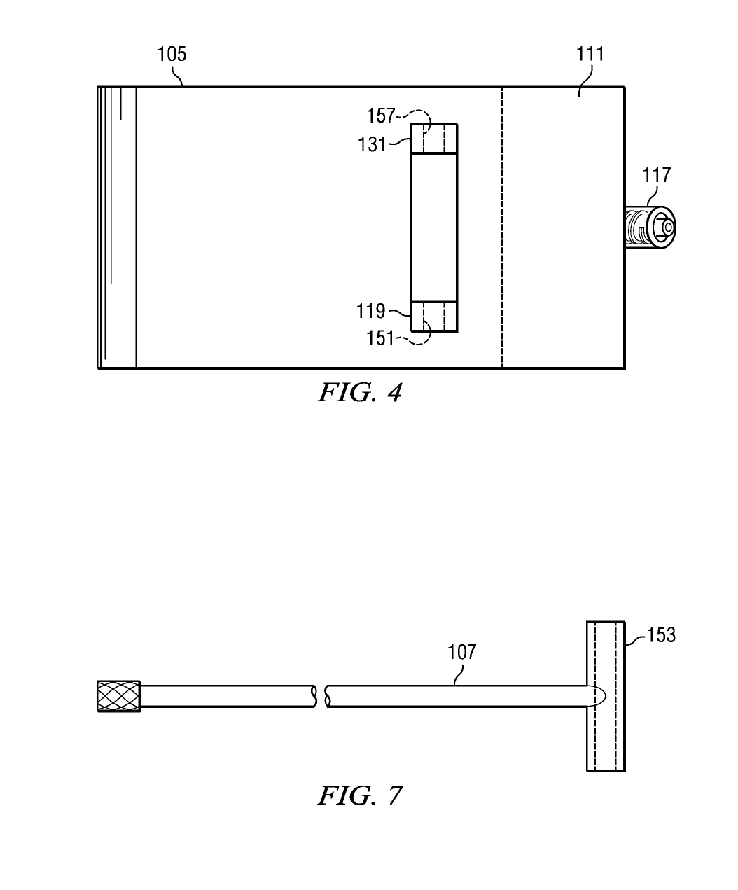

FIG. 4 illustrates a top view of the drug delivery system 100 and illustrates the guide device which may include the first hinge portion 119 and the opposing second hinge portion 131 which may be attached to the top surface of the guide device 105. The first end portion 119 and the second hinge portion 113 includes a aperture 115 to cooperate with a cylinder portion 153 of the handle 107 so that the handle 107 may pivot in order to guide the drug delivery system 100. FIG. 4 illustrates the base portion 111 and the connector 117.

FIG. 7 illustrates a handle 107 which may be adapted to cooperate with the guide device 105 includes a cylinder portion 153. Any number of designs of the handle 107 may be used to support the injection sled 105.

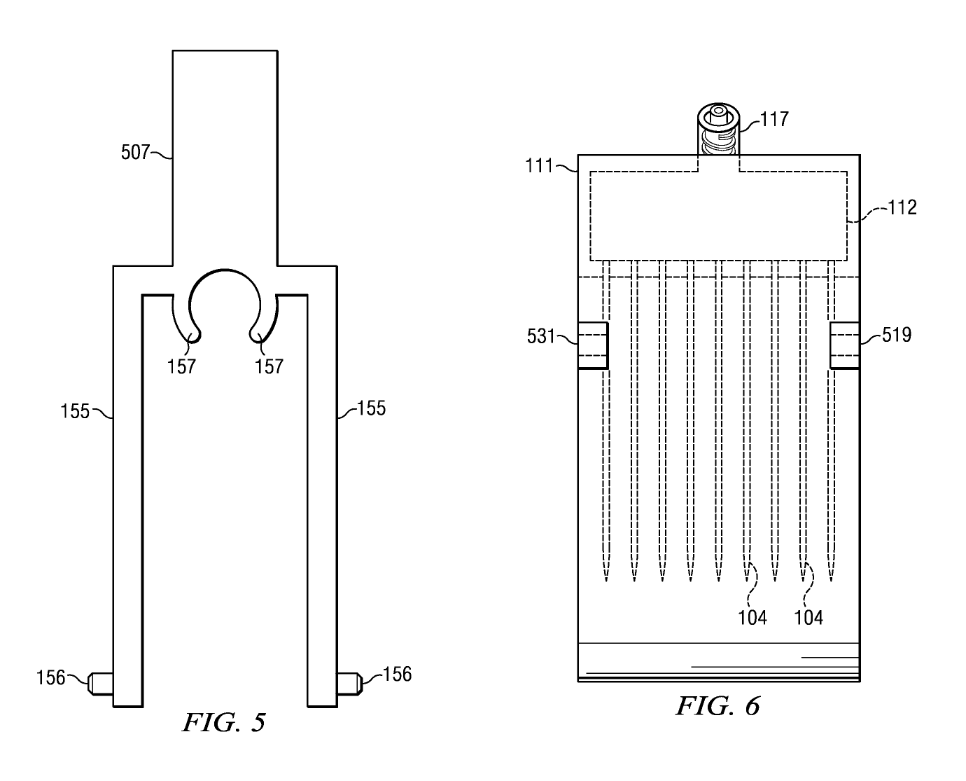

FIG. 5 illustrates an alternate handle 507 which may include a pair of opposing arms 155 which may include outward extending cylinders 156 and which may include a pair of opposing jaws 159 to cooperate and secure the flexible tubing 113.

FIG. 6 illustrates a first hinge portion 519 and a second hinge portion 531 to cooperate with the cylinders 156 so that the handle 507 can pivot. FIG. 6 additionally illustrates the connector 117, the central cavity 112 of the base portion 111 and the hollow cylinders 104 to deliver the drug to the patient.

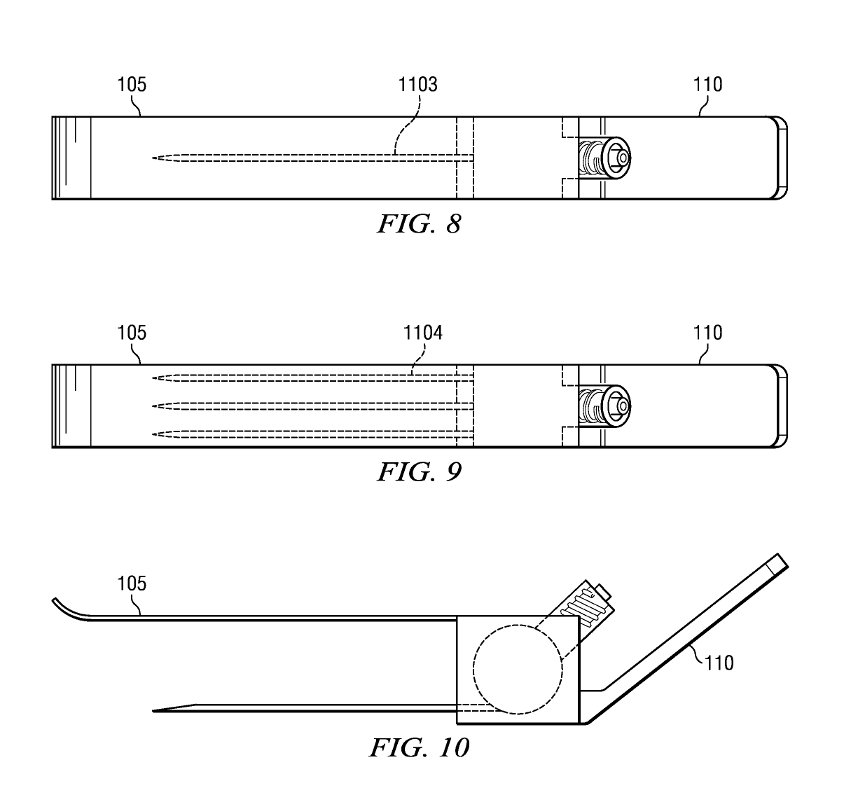

FIG. 8 illustrates a guide device 105 which may include a single needle 1103.

FIG. 9 illustrates a guide device 105 which may include multiple needles 1104.

FIG. 10 illustrates a guide device 105 which may include an angled handle 1107.

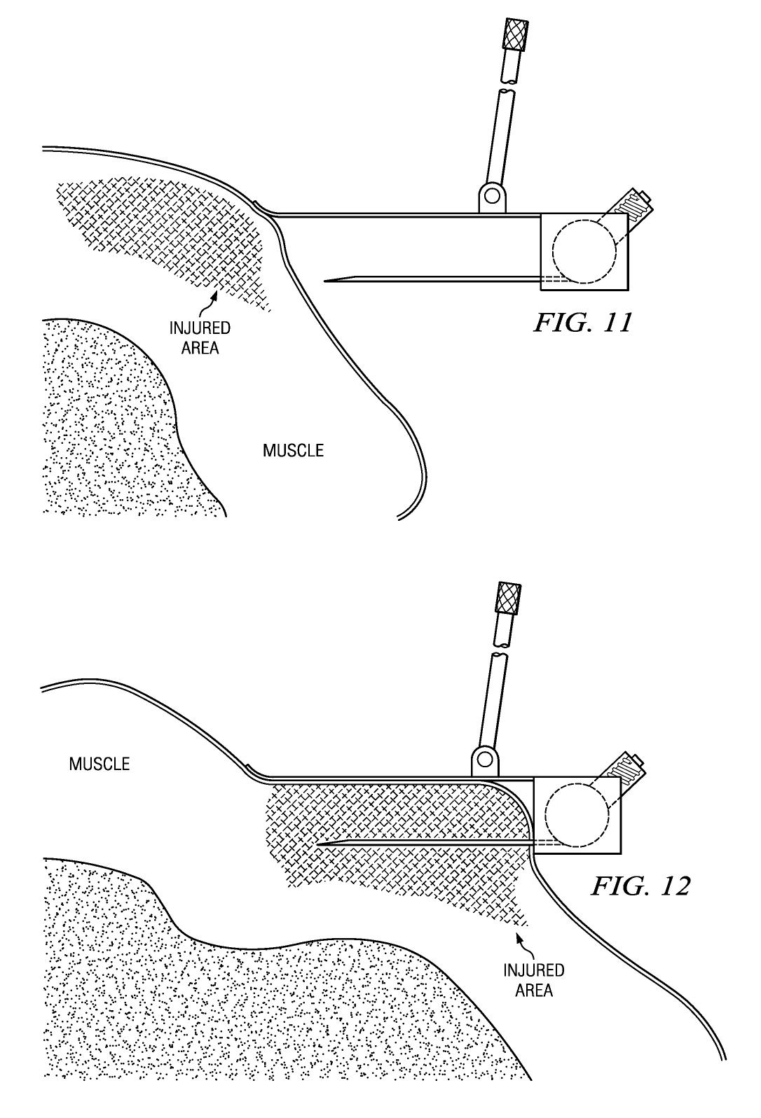

FIG. 11 illustrates the guide device 105 in a first position being adjacent to an injured area.

FIG. 12 illustrates the guide device 105 in a second position being positioned within the injured area.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A drug delivery system for delivering a drug, comprising:
a needle array to deliver the drug or biological materials to the patient;
a guide device to guide and control the depth and distance of penetration into the target tissue of the needle array;
a base portion to connect the needle array to the guide device.

2) A drug delivery system for delivering a drug as in claim 1, wherein the needle array includes a single row of needles.

3) A drug delivery system for delivering a drug as in claim 1, wherein the guide device includes a concave end.

4) A drug delivery system for delivering a drug as in claim 1, wherein the base portion includes a connector.

5) A drug delivery system for delivering a drug as in claim 4, wherein the connector includes a luer type connector.

6) A drug delivery system for delivering a drug as in claim 1, wherein the needle array includes multiple rows of needles.
